(19) United States
(12) Patent Application Publication
(10) Pub. No.: US 2026/0036581 A1
(43) Pub. Date: Feb. 5, 2026

(54) SPATIAL CHARACTERIZATION OF THE FECAL MICROBIOME

(71) Applicant: Kanvas Biosciences, Inc., Monmouth Junction, NJ (US)

(72) Inventors: Prateek Sehgal, Monmouth Junction, NJ (US); Gregory Booth, Monmouth Junction, NJ (US); Philip Burnham, Monmouth Junction, NJ (US); Matthew Cheng, Monmouth Junction, NJ (US); Kevin Cutler, Monmouth Junction, NJ (US); Iwijn De Vlaminck, Monmouth Junction, NJ (US); Anjali Doshi, Monmouth Junction, NJ (US); Kyle Jacoby, Monmouth Junction, NJ (US); Gaetano Scuderi, Monmouth Junction, NJ (US); Hao Shi, Monmouth Junction, NJ (US); Lee Swem, Monmouth Junction, NJ (US); Maya Trisolini, Monmouth Junction, NJ (US)

(73) Assignee: Kanvas Biosciences, Inc., Monmouth Junction, NJ (US)

(21) Appl. No.: 19/288,083

(22) Filed: Aug. 1, 2025

Related U.S. Application Data
(60) Provisional application No. 63/678,339, filed on Aug. 1, 2024.

(51) Int. Cl. G01N 33/569; A61K 35/74

(52) U.S. Cl. CPC G01N 33/56911; A61K 35/74; G01N 2333/19

(57) ABSTRACT

Disclosed herein is a method of evaluating the fecal microbiome of a subject comprising determining a spatial characterization of the fecal microbiome.

### PRIORITY CLAIM

This application claims priority to U.S. Provisional Patent Application No. 63/678,339, filed Aug. 1, 2024, which is incorporated by reference in its entirety.

### BACKGROUND

Studies have demonstrated that the composition of the microbiome, measured by sequencing, microbial culture or quantitative polymerase chain reaction (qPCR), offers valuable insights into patient health across various conditions, including the existence of, or vulnerability to, infectious diseases, autoimmune disorders, and gastrointestinal (GI) issues and conditions such as inflammatory bowel disease.

In addition, the potential for introducing complementary microbiomes in patients for a variety of therapeutic and prophylactic purposes is gaining interest in the scientific and medical communities. While some studies show modest success, the need to improve the proportion of positive outcomes remains vital.

There is a need for biomarker development which could be used to improve the understanding of patient health and to select an appropriate therapy, e.g., complementary microbiome therapy. Better patient selection tools could improve response rates and ensure patients are on the most effective therapeutics for their specific condition as early as possible.

### SUMMARY

In one aspect, a method of evaluating the fecal microbiome of a subject can include determining a spatial characterization of the fecal microbiome.

In another aspect, a method of identifying fecal microbiome biomarkers for responding to a treatment can include: a) transplanting fecal material from a treatment responder to a laboratory animal; b) allowing time for engraftment; and c) examining gastrointestinal tissue in the animal for the appearance of spatial markers as compared to control.

In some embodiments, determining the spatial characterization of the fecal microbiome can include: a) imaging a fecal specimen that has been fixed, permeabilized, hybridized with encoding and readout probes, and, optionally, stained; and b) processing the imaging data to segment and characterize microbes.

In some embodiments, the spatial characterization of the fecal microbiome can include measuring of one or more of microbial spatial structure, microbial spatial location, microbial intensity, microbial packing, microbial neighborhood composition, microbial interaction matrix, microbial spatial diversity, gene expression measure of microbial cell states and microbial morphology.

In some embodiments, the spatial characterization of the fecal microbiome can be compared between a healthy subject and a subject with a disease.

In some embodiments, the spatial characterization of the fecal microbiome can be compared between a patient who responds to treatment for a disease and a patient who does not respond to the treatment.

In some embodiments, the spatial characterization of the fecal microbiome can be conducted in a patient to determine the appropriate treatment. In certain circumstances, the treatment, or appropriate treatment, can be complementary microbiome treatment.

In some embodiments, the spatial characterization of the fecal microbiome can be conducted to assess the efficacy of the treatment. In certain circumstances, the treatment can be complementary microbiome treatment, for example, the complementary microbiome treatment can include a microbial consortium.

Other aspects, embodiments, and features will be apparent from the following description, the drawings, and the claims. All references cited herein, including patent applications, patent publications, UniProtKB accession numbers, are herein incorporated by reference, as if each individual reference were specifically and individually indicated to be incorporated by reference in its entirety.

### BRIEF DESCRIPTION OF THE DRAWINGS

FIG. 1 is a diagram of measurements using imaging-based platforms applied to microbiome samples as imaged by microscopy. Microbiota are colored by taxonomic identity (black, white, patterned); host cells (H.C.) are light and medium gray; food particles (F.P.) are dark gray. The larger microscope image on the left has a dotted line square which depicts what is zoomed-in for further Neighborhood (e.g., local Shannon diversity), Network-based (e.g., inter-taxa proximity), Morphometric (e.g., area, solidity), and Landmark-based (e.g., distance to host cells and food particles) analysis.

FIG. 2 (panels A-E) shows graphs of neighborhood-based measurements for different neighborhood sizes measuring Shannon Diversity, Bacterial Density, and Taxa Richness as noted, comparing different properties. FIG. 2, panel A compares microbiomes with high (n=50) and low (n=10) number of taxa, FIG. 2, panel B compares uniform and power-law distributed relative abundance (uniform vs. skewed abundance), FIG. 2, panel C compares dense and sparse (25% of full density) microbial communities, FIG. 2, panel D compares randomly mixed and clustered communities (using k-means clustering to create like-taxa to be nearer to each other), and FIG. 2, panel E shows a mixture of multiple conditions (skewed, dense, random mixing vs uniform, sparser, high clustering).

FIG. 3A shows a proximity network constructed from real microbiome imaging data. A micrograph of interacting microbiota is shown, a network of edges is shown superimposed over bacteria that fall within 1 micron of one another. FIG. 3B shows a correlated spatial association matrix. The relative spatial association (RSA) was calculated for each species. Darker squares indicate more associations.

FIGS. 4A-4C are diagrams showing the principles of constructing the spatial association matrix. Higher numbers of asterisks (*) indicate more associations. FIG. 4A shows the measured environment, FIG. 4B shows one random environment and FIG. 4C shows an alternative random environment.

FIG. 5 represents a microscopic image of microbiome from a mouse GI section showing centroids of individual microbes near host tissue, separated by a mucus layer.

FIGS. 6A-6B show further spatial organization of microbiome with respect to mouse host tissue. FIG. 6A displays histograms showing abundance distribution of two taxa (BCA071 on the left and VER008 on the right) in the mouse tissues ileum, cecum and colon, as well as distance from the host tissue. FIG. 6B is a bubble plot depicting the median distance of each taxon from host tissue alongside their abundances.

FIG. 7 shows measures of self-interactions of different taxa long the mouse GI tract. The images for each strain show spatial distribution of self-clusters for several individual taxa throughout the GI tract. Dark gray color indicates microbes that form clusters, while light gray represents microbes that do not.

FIGS. 8A-8B also show measures of self-interaction of different taxa along the mouse GI tract. In FIG. 8A (top), self-clustering strength is defined by cells that form clusters normalized by absolute abundance (C-Strength) for each taxon in each tissue. This analysis provides a direct representation of species-specific self-aggregation as observed in the images. In FIG. 8A (bottom), C-strength in the top heatmap is normalized by the C-strength measured from 5000 random simulations. In FIG. 8B, bar graphs show normalized clustering strength aggregated across all strains in each region of the GI tract. The normalized clustering tendency is decreased along the GI tract, which is consistent with the increasing homogenization of fecal matter as it progresses through the gut.

FIGS. 9A-9B show measures of cluster organization and ribosomal intensity along the mouse GI tract. In FIG. 9A, the median distance of identified clusters of a taxon from the host tissue is shown, normalized by the median distance of all microbes of that taxon from the host tissue. The color of the disk represents distance and the size represents abundance. Only taxa that form clusters in all three regions of GI tract are shown for comparison. A value of −1 indicates that the taxon forms clusters closer to the host compared to its overall distribution, while a value of +1 indicates that the taxon forms clusters farther from the host relative to its overall distribution. FIG. 9B shows a comparison of ribosomal intensity between cells that are within clusters and those outside of clusters, analyzed by taxon and tissue type. Lighter color indicates taxa where clustered cells have higher ribosomal intensity than non-clustered cells, while darker color indicates the opposite. The color bar extrema are capped at −50% to +50% for visualization purposes. Taxa that do not form any self-clusters are assigned a zero value.

FIG. 10 provides diagrams depicting measurement of cross-species interactions of various taxa at microscale and show non-random structures. Chord-style description shows absolute (“All Interactions” at top) and significant interactions (“Significant” at bottom) across all measured taxa in each tissue, ileum, cecum and colon. Self-interactions are excluded. Light gray chords show positive associations, while dark gray chords show negative associations. The significance is assessed by comparing the associations against randomized versions of the region adjacency graph.

FIGS. 11A-11B provide images of cross-species interactions at mesoscale and show non-random structures. FIG. 11A demonstrates results, for mesoscale interactions, where disks ranging from radius 25 μm to 250 μm were generated and the density of each taxon in each tissue was used to generate microbiome profiles. FIG. 11A (left) shows the disk profiles projected into a 2D space using UMAP and a KNN and Leiden clustering approach which was used to generate distinct neighborhoods. FIG. 11A (right) shows the frequency of each of these neighborhoods. FIG. 11B details the distribution of neighborhoods projected onto radius −75 μm disks in each tissue. Notably, each tissue type contained a unique set of neighborhoods, highlighting the heterogeneity of spatial organization between these tissues at the mesoscale.

FIG. 12 shows the characteristics of the neighborhoods including microbial load (taxa per 10,000 μm²), relative abundances of different taxa.

FIGS. 13A-13B show cross-species interactions at mesoscale with non-random structures. FIG. 13A details the characteristics of the neighborhoods including relative disk-size frequency, relative frequency per tissue, and distance from host. FIG. 13B shows the significance of neighborhoods over random distributions. Identity randomization was used to examine neighborhood reassignment. Percentage change indicates the change in neighborhood frequency upon randomization. Strong positive values indicate neighborhoods with strong, nonrandom spatial structures.

FIGS. 14A-14B detail measurements of human clinical samples showing differences between taxon abundance and taxon association. FIG. 14A shows metagenomic profiling of five major bacterial phyla across a clinical-trial cohort. Ten representative samples were selected for whole-metagenome sequencing and analyzed with MetaPhlAn; relative phylum abundances were then aggregated for each sample. FIG. 14B shows HiPR-Map fluorescence imaging targeting the same five phyla and was performed on 0.07 mm² regions of each specimen. Spatial adjacency graphs were constructed to count inter-phylum contacts; shading in the heatmap reflects interaction frequency for a subset of the ten samples.

FIGS. 15A-15B show measurements of clinical samples with differences between taxon abundance and taxon association. Principal coordinates analysis (PCoA) of the two datasets—relative-abundance profiles (FIG. 15A) and vectorized phylum-phylum interaction matrices (FIG. 15B)—reveals that samples clustering by overall abundance do not always cluster similarly when spatial interaction patterns are considered.

### DETAILED DESCRIPTION

Specific microbiome characteristics, such as alpha diversity or beta diversity in relation to a drug product, can direct the administration of targeted therapies, including microbiome treatments, such as with fecal microbiota transplants (FMTs) or live biotherapeutic products (LBPs), as well as the administration of other biologics or small molecule therapeutics. Additionally, the microbiome can provide general markers regarding patient health.

Advanced spatial biology platform and imaging technologies provide a unique and potent means to additionally evaluate patients and, potentially, tailor treatment strategies. A diverse range of microbiome characteristics, including bacterial cell morphology, the spatial relationships between cells and non-bacterial entities (such as food particles, other noncellular organic materials, and host cells), as well as larger community structures, self-similarity, and stratification of taxa around landmarks (e.g. the edge of the feces vs. the core), can provide a comprehensive understanding of how the spatial characteristics of the fecal microbiome influence human health and response to treatment, how human health and disease can impact the microbiome, and also how microbiome characteristics can help identify the appropriate targeted microbiome therapy.

### Definitions

By “fecal microbiome” of a subject is meant the community of microorganisms that exist in a fecal specimen from the subject.

By “spatial characterization of the fecal microbiome” is meant characterizing the fecal microbiome for one or more of the community structure of microbial phylogeny (e.g., taxa or species), the microbial spatial relationships between each other and non-microbe entities, such as food particles, other noncellular organic materials and host cells, self-similarity (an arbitrarily small portion of the sample looks similar to a bigger piece or the whole sample) and stratification of microbial phylogeny within fecal material around landmarks such as fecal edge vs. core.

By “microbial spatial structure” is meant the self-organization of microbes as determined by the structure measurement metrics such as the structure factor to identify states such as hyperuniformity.

By “microbial spatial location” is meant the spatial location of microbes in the microbiome relative to the overall sample or medium in which the microbiome is embedded.

By “microbial intensity” is meant a measure of microbe function.

By “microbial packing” is meant the spatial density of a microbe.

By “microbial neighborhood composition” is meant the composition of microbes within a certain radial distance from a base microbe or fiducial in the sample within the medium. It also encompasses the local network of microbes connected by edges, wherein the connected nodes represent microbes within a specific physical distance from each other.

By “microbial interaction matrix” is meant the tabulated frequency of edges between each taxon in a graph representing physical proximity in the experiment.

By “microbial spatial diversity” is meant a determination of whether similar microbes at the phylogenetic level occur near each other.

By “gene expression of microbial cell state” is meant the measure of gene expression of microbial states such as stress response, biofilm production, quorum sensing, and antimicrobial resistance.

By “microbial morphology” is meant determination of microbial size and shape and the overall diversity of these characteristics with regard to each other.

By “complementary microbiome” is meant microbes that are present in a favorable spatial system in the fecal material from a donor, e.g., fecal material from a healthy donor, fecal material from a treatment responder, a super donor, or in a therapeutic microbial consortium.

By “microbial consortium” is meant a mixture of two or more isolated strains of microbes that have been cultured.

By “super donor” is meant a subject that responds to a treatment and who has a favorable property in their fecal material that converts another subject who is a non-responder to the treatment into a responder after FMT from the super donor.

By “curated microbial consortium” is a microbial consortium wherein the strains are selected based upon strains present, for example, in a donor fecal sample.

By “fecal microbiota” is meant a mixture of two or more bacterial strains derived from human fecal material.

As used herein, the terms “gastrointestinal engraftment” or “engraft” or “engraftment” refers to the establishment of one or more than one microbe, or microbial consortium, in one or more than one niche of the gastrointestinal tract that, prior to administration of the microbial consortium, was not present or detectable. Engraftment may be transient or may be persistent.

As used herein, the term “supportive community” refers to one or more than one microbial strain that, when administered with an active microbe, enhances one or more than one characteristic of the active microbe selected from the group consisting of gastrointestinal engraftment, biomass, metabolic substrate metabolism, and longitudinal stability.

As used herein, the term “pharmaceutical composition” refers to the combination of an active agent with a carrier, inert or active, making the composition especially suitable for therapeutic use in vivo or ex vivo.

As used herein, the term “pharmaceutically acceptable carrier” refers to any of the standard pharmaceutical carriers, such as phosphate buffered saline solution, water, emulsions (e.g., oil/water or water/oil emulsions), and various types of wetting agents. The compositions also can include stabilizers and preservatives. For examples of carriers, stabilizers, and adjuvants, see e.g., Martin, Remington's Pharmaceutical Sciences, 15th Ed. Mack Publ. Co., Easton, Pa. [1975].

### Microbial Consortium

The methods described herein can include the use of a complementary microbiome. In some cases, the complementary microbiome can be in the form of a microbial consortium. A microbial consortium is comprised of a plurality of active microbes and an effective amount of a supportive community of microbes. In some embodiments, a microbial consortium comprises 2 to 500 microbial strains. For example, in some embodiments, a microbial consortium comprises 3 to 500, 4 to 500, 5 to 500, 6 to 500, 7 to 500, 8 to 500, 9 to 500, 10 to 500, 11 to 500, 12 to 500, 13 to 500, 14 to 500, 15 to 500, 16 to 500, 17 to 500, 18 to 500, 19 to 500, 20 to 500, 21 to 500, 22 to 500, 23 to 500, 24 to 500, 25 to 500, 30 to 500, 35 to 500, 40 to 500, 45 to 500, 50 to 500, 60 to 500, 70 to 500, 80 to 500, 90 to 500, 100 to 500, 110 to 500, 120 to 500, 130 to 500, 140 to 500, 150 to 500, 160 to 500, 170 to 500, 180 to 500, 190 to 500, 200 to 500, 210 to 500, 220 to 500, 230 to 500, 240 to 500, 250 to 500, 260 to 500, 270 to 500, 280 to 500, 290 to 500, 300 to 500, 400 to 500, 3 to 300, 4 to 300, 5 to 300, 6 to 300, 7 to 300, 8 to 300, 9 to 300, 10 to 300, 11 to 300, 12 to 300, 13 to 300, 14 to 300, 15 to 300, 16 to 300, 17 to 300, 18 to 300, 19 to 300, 20 to 300, 21 to 300, 22 to 300, 23 to 300, 24 to 300, 25 to 300, 30 to 300, 35 to 300, 40 to 300, 45 to 300, 50 to 300, 60 to 300, 70 to 300, 80 to 300, 90 to 300, 100 to 300, 110 to 300, 120 to 300, 130 to 300, 140 to 300, 150 to 300, 160 to 300, 170 to 300, 180 to 300, 190 to 300, 200 to 300, 210 to 300,220 to 300, 230 to 300, 240 to 300, 250 to 300, 260 to 300, 270 to 300, 280 to 300, 290 to 300, 3 to 250, 4 to 250, 5 to 250, 6 to 250, 7 to 250, 8 to 250, 9 to 250, 10 to 250, 11 to 250, 12 to 250, 13 to 250, 14 to 250, 15 to 250, 16 to 250, 17 to 250, 18 to 250, 19 to 250, 20 to 250, 21 to 250, 22 to 250, 23 to 250, 24 to 250, 25 to 250, 30 to 250, 35 to 250, 40 to 250, 45 to 250, 50 to 250, 60 to 250, 70 to 250, 80 to 250, 90 to 250, 100 to 250, 110 to 250, 120 to 250, 130 to 250, 140 to 250, 150 to 250, 160 to 250, 170 to 250, 180 to 250, 190 to 250, 200 to 250, 210 to 250, 220 to 250, 230 to 250, 240 to 250, 3 to 200, 4 to 200, 5 to 200, 6 to 200, 7 to 200,8 to 200, 9 to 200, 10 to 200, 11 to 200, 12 to 200, 13 to 200, 14 to 200, 15 to 200, 16 to 200, 17 to 200, 18 to 200, 19 to 200, 20 to 200, 21 to 200, 22 to 200, 23 to 200, 24 to 200, 25 to 200, 30 to 200, 35 to 200, 40 to 200, 45 to 200, 50 to 200, 60 to 200, 70 to 200, 80 to 200, 90 to 200, 100 to 200, 110 to 200, 120 to 200, 130 to 200, 140 to 200, 150 to 200, 160 to 200, 170 to 200, 180 to 200, 190 to 200, 3 to 150,4 to 150, 5 to 150, 6 to 150, 7 to 150, 8 to 150, 9 to 150, 10 to 150, 11 to 150, 12 to 150, 13 to 150, 14 to 150, 15 to 150, 16 to 150, 17 to 150, 18 to 150, 19 to 150, 20 to 150, 21 to 150, 22 to 150, 23 to 150, 24 to 150, 25 to 150, 30 to 150, 35 to 150, 40 to 150, 45 to 150, 50 to 150, 60 to 150, 70 to 150, 80 to 150, 90 to 150, 100 to 150, 110 to 150, 120 to 150, 130 to 150, 140 to 150, 3 to 100, 4 to 100, 5 to 100, 6 to 100, 7 to 100, 8 to 100, 9 to 100, 10 to 100, 11 to 100, 12 to 100, 13 to 100, 14 to 100, 15 to 100, 16 to 100, 17 to 100, 18 to 100, 19 to 100, 20 to 100, 21 to 100, 22 to 100, 23 to 100, 24 to 100, 25 to 100, 30 to 100, 35 to 100, 40 to 100, 45 to 100, 50 to 100, 60 to 100, 70 to 100, 80 to 100, 90 to 100, 3 to 75, 4 to 75, 5 to 75, 6 to 75, 7 to 75, 8 to 75, 9 to 75, 10 to 75, 11 to 75, 12 to 75, 13 to 75, 14 to 75, 15 to 75, 16 to 75, 17 to 75, 18 to 75, 19 to 75, 20 to 75, 21 to 75, 22 to 75, 23 to 75, 24 to 75, 25 to 75, 30 to 75, 35 to 75, 40 to 75, 45 to 75, 50 to 75, 60 to 75, 70 to 75, 3 to 50, 4 to 50, 5 to 50, 6 to 50, 7 to 50, 8 to 50, 9 to 50, 10 to 50, 11 to 50, 12 to 50, 13 to 50, 14 to 50, 15 to 50, 16 to 50, 17 to 50, 18 to 50, 19 to 50, 20 to 50, 21 to 50, 22 to 50, 23 to 50, 24 to 50, 25 to 50, 30 to 50, 35 to 50, 40 to 50, 45 to 50, 3 to 25, 4 to 25, 5 to 25, 6 to 25, 7 to 25, 8 to 25, 9 to 25, 10 to 25, 11 to 25, 12 to 25, 13 to 25, 14 to 25, 15 to 25, 16 to 25, 17 to 25, 18 to 25, 19 to 25, 20 to 25, 21 to 25, 22 to 25, 23 to 25, or 24 to 25 microbial strains. For example, in some embodiments, a microbial consortium comprises about 20 to about 200, about 70 to about 80, about 80 to about 90, about 100 to about 110, or about 150 to about 160 microbial strains.

In some embodiments, a microbial consortium described herein comprises a microbial strain having a relative abundance of approximately 90%, 80%, 70%, 60%, 50%, 40%, 30%, 20%, 10%, 5%, 1%, 0.1%, 0.01%, 0.001%, 0.0001%, 0.00001%, or 0.000001% of the total microbial consortium. In some embodiments, the relative abundance of a microbial strain is determined, for example, by metagenomic sequencing, High Phylogenetic Resolution microbiome mapping by Fluorescence in situ Hybridization (HiPR-FISH), quantitative PCR (qPCR), or droplet digital PCR, and calculated as the percentage of reads that are classified as an identified microbial strain, divided by the genome size or the counted number of bacterial of particular taxa divided by the total counted number of bacteria. For example, in some embodiments, the relative abundance of a microbial strain is determined, for example, by metagenomic shotgun sequencing, HiPR-FISH, or qPCR.

### Dosages

In some embodiments, a microbial consortium is administered in a dosage form having a total amount of microbial consortium of 0.1 ng to 500 mg, 0.5 ng to 500 mg, 1 ng to 500 mg, 5 ng to 500 mg, 10 ng to 500 mg, 50 ng to 500 mg, 100 ng to 500 mg, 500 ng to 500 mg, 1p g to 500 mg, 5 μg to 500 mg, 10 μg to 500 mg, 50 μg to 500 mg, 100 μg to 500 mg, 500 μg to 500 mg, 1 mg to 500 mg, 5 mg to 500 mg, 10 mg to 500 mg, 50 mg to 500 mg, 100 mg to 500 mg, 0.1 ng to 100 mg, 0.5 ng to 100 mg, 1 ng to 100 mg, 5 ng to 100 mg, 10 ng to 100 mg, 50 ng to 100 mg, 100 ng to 100 mg, 500 ng to 500 mg, 1 μg to 100 mg, 5 μg to 100 mg, 10 μg to 100 mg, 50 μg to 100 mg, 100 μg to 100 mg, 500 μg to 100 mg, 1 mg to 500 mg, 5 mg to 100 mg, 10 mg to 100 mg, 50 mg to 100 mg, 0.1 ng to 50 mg, 0.5 ng to 50 mg, 1 ng to 50 mg, 5 ng to 50 mg, 10 ng to 50 mg, 50 ng to 50 mg, 100 ng to 50 mg, 500 ng to 500 mg, 1 μg to 50 mg, 5 μg to 50 mg, 10 μg to 50 mg, 50 μg to 50 mg, 100 μg to 50 mg, 500 μg to 50 mg, 1 mg to 500 mg, 5 mg to 50 mg, 10 mg to 50 mg, 0.1 ng to 10 mg, 0.5 ng to 10 mg, 1 ng to 10 mg, 5 ng to 10 mg, 10 ng to 10 mg, 50 ng to 10 mg, 100 ng to 10 mg, 500 ng to 500 mg, 1 μg to 10 mg, 5 μg to 10 mg, 10 μg to 10 mg, 50 μg to 10 mg, 100 μg to 10 mg, 500 μg to 10 mg, 1 mg to 500 mg, 5 mg to 10 mg, 0.1 ng to 5 mg, 0.5 ng to 5 mg, 1 ng to 5 mg, 5 ng to 5 mg, 10 ng to 5 mg, 50 ng to 5 mg, 100 ng to 5 mg, 500 ng to 500 mg, 1 μg to 5 mg, 5 μg to 5 mg, 10 μg to 5 mg, 50 μg to 5 mg, 100 μg to 5 mg, 500 μg to 5 mg, 1 mg to 500 mg, 0.1 ng to 1 mg, 0.5 ng to 1 mg, 1 ng to 1 mg, 5 ng to 1 mg, 10 ng to 1 mg, 50 ng to 1 mg, 100 ng to 1 mg, 500 ng to 500 mg, 1 μg to 1 mg, 5 μg to 1 mg, 10 μg to 1 mg, 50 μg to 1 mg, 100 μg to 1 mg, 500 μg to 1 mg, 0.1 ng to 500 μg, 0.5 ng to 500 μg, 1 ng to 500 μg, 5 ng to 500 μg, 10 ng to 500 μg, 50 ng to 500 μg, 100 ng to 500 μg, 500 ng to 500 μg, 1 μg to 500 μg, 5 μg to 500 μg, 10 μg to 500 μg, 50 μg to 500 μg, 100 μg to 500 μg, 0.1 ng to 100 μg, 0.5 ng to 100 μg, 1 ng to 100 μg, 5 ng to 100 μg, 10 ng to 100 μg, 50 ng to 100 μg, 100 ng to 100 μg, 500 ng to 100 μg, 1 μg to 100 μg, 5 μg to 100 μg, 10 μg to 100 μg, 50 μg to 100 μg, 0.1 ng to 50 μg, 0.5 ng to 50 μg, 1 ng to 50 lag, 5 ng to 50 μg, 10 ng to 50 μg, 50 ng to 50 μg, 100 ng to 50 μg, 500 ng to 50 μg, 1 μg to 50 μg, 5 μg to 50 μg, 10 μg to 50 μg, 0.1 ng to 10 μg, 0.5 ng to 10 μg, 1 ng to 10 μg, 5 ng to 10 μg, 10 ng to 10 μg, 50 ng to 10 μg, 100 ng to 10 μg, 500 ng to 10 μg, 1 μg to 10 μg, 5 μg to 10 μg, 0.1 ng to 5 μg, 0.5 ng to 5 μg, 1 ng to 5 μg, 5 ng to 5 μg, 10 ng to 5 μg, 50 ng to 5 μg, 100 ng to 5 μg, 500 ng to 5 μg, 1 μg to 5 μg, 0.1 ng to 1 μg, 0.5 ng to 1 μg, 1 ng to 1 μg, 5 ng to 1 μg, 10 ng to 1 μg, 50 ng to 1 μg, 100 ng to 1 μg, 500 ng to 1 μg, 0.1 ng to 500 ng, 0.5 ng to 500 ng, 1 ng to 500 ng, 5 ng to 500 ng, 10 ng to 500 ng, 50 ng to 500 ng, 100 ng to 500 ng, 0.1 ng to 100 ng, 0.5 ng to 100 ng, 1 ng to 100 ng, 5 ng to 100 ng, 10 ng to 100 ng, 50 ng to 100 ng, 0.1 ng to 50 ng, 0.5 ng to 50 ng, 1 ng to 50 ng, 5 ng to 50 ng, 10 ng to 50 ng, 0.1 ng to 10 ng, 0.5 ng to 10 ng, 1 ng to 10 ng, 5 ng to 10 ng, 0.1 ng to 5 ng, 0.5 ng to 5 ng, 1 ng to 5 ng, 0.1 ng to 1 ng, 0.1 ng to 1 ng, or 0.1 ng to 0.5 ng total dry weight.

In some embodiments, a microbial consortium is administered as a single dose or as multiple doses. In some embodiments, a microbial consortium is administered intermittently. For example, in some embodiments, a microbial consortium is administered once weekly, once monthly, or when a subject is in need thereof. In certain embodiments, the consortium is administered once a day for 2 days, 3 days, 4 days, 5 days, 6 days, or 1 week. In certain embodiments, the consortium is administered once a week for 2 weeks, 3 weeks, or 1 month. In some embodiments, the consortium is administered once a month for 2 months, 3 months, 4 months, 5 months, 6 months, or 1 year.

In certain embodiments, the consortium is administered at an effective dose to allow for engraftment.

### Examples

The disclosure now being generally described, will be more readily understood by reference to the following examples which are included merely for purposes of illustration of certain aspects and embodiments of the present disclosure and are not intended to limit the scope of the disclosure in any way.

Here we reference species and taxon interchangeably. Species is a particular phylogenetic level where taxon is more broadly defined. While taxon and species are used herein, it is intended that all levels of phylogeny are measured and assessed using these methods.

### Example 1. General

All analysis described here follows these general steps: the specimen is fixed; permeabilized; hybridized with encoding probes; hybridized with readout probes; optionally stained (e.g. DAPI); mounted; and imaged (e.g., confocal, lightsheet).

Following imaging, the data is processed, comprising: (1) generating segmented microbial objects and (2) assigning a taxonomic identity to the microbial objects in the specimen. Identities are related to the objects in the images, as well as individual and ensemble measurements, for example, as related to morphology and neighborhood properties.

### Example 2. Determination of the Presence or Absence of Microbes

Determination of presence/absence of taxa in the sample is measured, for example, if >n microbes of that taxa are detected over x images of the sample, then it is marked as present.

### Example 3. Direct Neighbor/Interaction Measurement

In this measure, for example, Species A is near Species B at some significant frequency.

The frequency of contacts between each species and every other species is assessed. This is an interaction matrix. Metrics could be drawn from networks built from the physical proximity of microbes. Then, species identities are randomly redistributed to the image masks and the interaction matrix is recalculated. This is repeated a large number of times (e.g., n=10000), the average is calculated for the random interaction network. Then this value is compared to the measured interaction network to look for significance. See, for example, PCT Application No. PCT/US24/22443, filed Apr. 1, 2024, which is incorporated by reference in its entirety.

The microbial interaction matrix can be compared to a random reassortment of taxonomic identifications in the same network by dividing the measured microbial interaction matrix by the randomly generated interaction matrix; this is referred to as “spatial association matrix.”

Other metrics include contiguity of a species and the proximity index of a species to another species.

As an example of assessing contiguity of a species, the largest area made of a particular taxon physically interacting with itself or the size of the continuous sub-network of microbes within a proximity network belonging to the same taxa is determined. Imagining a line of ten E. coli touching each other on the poles, the area would be 10× the area of a single E. coli bacterium.

As an example of assessing the proximity index of a species to another species, “buffer” windows are drawn around each bug (for example, a 3 μm buffer window); the average overlap of buffer zones between each species and every other species is then determined.

It is also possible to assess the number of unique species spatially located between two microbes of the same species/taxa. That is, microbe A may be determined to typically have two other taxa of microbes between it and another microbe A, whereas microbe B may be determined to have typically four or more other taxa of microbes between it and another microbe B.

### Example 4. Spatial Structure Measurements

The self-organization of microbes is quantified by measuring signatures of hyperuniformity, that is, a hidden order in a seemingly disordered system at larger length scales.

Two metrics are calculated for each taxa as well as all taxa combined: Number variance

$$( {σ}_{N}^{2} )$$

as a function of observation window size R; Structure factor S(k) as a function of spatial wave number k.

It is quantified if

$${σ}_{N}^{2}$$

scales slower than the volume of window size and if S(k) vanishes at smaller wavenumbers. If so, then the microbiome or taxon is said to possess an organized structure at larger length scales (hyperuniform).

We correlate this measured organization or lack thereof with the different states of microbiome present in healthy and diseased samples as well as in treatment responders vs. treatment nonresponders.

The overarching idea is that the interaction of microbes with microbes, and microbes with host (host cells, structures, food particles), leads to spatial patterns that have a non-trivial structure in them and that these interactions are potentially governed by the healthy or diseased states of the host. The reverse can also be true, that is, these interactions could themselves result in different states of health and disease in the host. Therefore, quantification of these spatial structures can be used as a biomarker as described herein (e.g., healthy vs. diseased, or treatment responders vs. nonresponders) (for more on hyperuniformity, see, e.g., Yang et al., Phys Rev E Stat Nonlin Soft Matter Phys 89(2): 022721, Feb 2014; Torquato, Physics Reports 745: 1-95, 6 Jun. 2018; Zheng et al., Soft Matter 16: 5942-5950, 2020.)

The power law relationship between spatial scale (e.g., area or volume) and diversity metric (e.g., Simpson's index, reverse Simpson's index, alpha diversity) has previously been found to stratify diseased and healthy per-implant oral microbial populations (Grodner et al., BioRxiv, July 2024). Such a relationship can be measured from stool to prescreen for patients likely to benefit from microbiome therapy.

### Example 5: Spatial Location Measurements

Here the spatial location of taxa in the microbiome is measured relative to the sample or medium in which the microbiome is embedded. In one example, Taxon A is enriched in the periphery of the stool, while Taxon B is enriched in the core of the stool.

### Example 6: Intensity Measurements

Here the intensity of a taxon is used as a measure of its activity in the gut. Such activity could be measured, for example, by HiPR-FISH. The brighter microbes of a given taxon are more active in their function relative to the dimmer microbes of that taxon. Exemplary ways to quantify this include looking for bimodal signal distribution within specific classified taxonomic groups. Also, the brightness of each taxa in each sample (e.g., healthy or diseased) can be compared and that relative ratio can be used as a biomarker in conjunction with other spatial metrics.

One can also assess heterogeneity of ribosomal RNA (rRNA) signal within each microbe or within each taxon population. By way of example, early indications from experiments show that stationary phase bacteria have more heterogeneous signal intensity within the taxon than that of exponential phase bacteria.

### Example 7: Packing Measurements

Once can use scaling of density of a taxon with the area of the measurement region as a biomarker with time or viability. As an example, Taxon A has a different spatial density profile relative to Taxon B.

One can also assess clustering of microbes as a proxy for measuring growth state. That is, if certain microbes are found in pairs or tetrads, they may have been dividing at the time of fixation.

One can also use the pair correlation function g(r) to quantify taxa distribution relative to each other. Linear dipole analysis can be used to quantify the tendency of two taxa to attract or repel each other (Wilbert et al., Cell Reports 30(12) 4003-4015.e3, 2020; Ramirez-Puebla et al., Microbiome 10: Art. 52, 2022).

### Example 8: Neighborhood Measurements

Here are neighborhood measures by way of example: 1) a circle/sphere with radius R contains X bacteria of species (taxon) A and Y bacteria of species (taxon) B; and 2) a circle/sphere with radius R contains X total different species (taxa) of bacteria. These metrics can be determined from a single measurement or summarized from multiple measurements, using methods such as a ‘rolling window’ approach, where a smaller window is moved across a larger dataset to capture local variations.

Over multiple images of the same sample, measure the relative percent presence of different taxa/species (i.e., in field of view (FoV) 1 or Region 1, 90% of the microbes are taxa A and 10% are taxa B, whereas in FoV2 or Region 2, 50% of the microbes are taxa A, 25% are taxa B, and 25% are taxa C.)

### Example 9: Spatial Diversity Measurements

Metrics that can be used to measure spatial diversity include: Moran's I measurement of spatial autocorrelation, indicating whether similar species (taxa) occur near each other; Geary's C measurement of global spatial autocorrelation; and Getis-Ord statistics measurements of global and local spatial autocorrelation. For example, proximity to host cells, or location within specific host-defined regions, e.g., crypts, could be important.

### Example 10: Morphological Measurements

For morphological metrics, any number of tools can be used to measure the size and shape of objects, and the overall diversity of these shapes with regards to each other.

Function can also be used as a measure, e.g., by gene expression for functions such as stress response, biofilm production, quorum sensing, antimicrobial resistance.

Fractal pattern measurements can be used. Elliptical Fourier Descriptors may be a basis for many metrics comparing between species and within a species. Also, with cell cluster morphology, segmented objects can be binned by species, taxa, etc. and morphological metrics can be performed on the group, e.g. microcolony/biofilm depth.

Distance field can be used as a general tool for measuring cell width, biofilm depth, etc. It can be used for calculating weighted averages of signal to reduce edge effects or to bin signal by distance from cell boundary (see intensity measurements and heterogeneity above.)

With morphological assessments, scaling/self-similarity among different species can be evaluated. Also, association with biofilms (which are detectable using particular stains) and whether biofilms associated with one taxa are also typically associated with another taxa can be evaluated.

Connect morphological measurements with spatial neighborhood analysis. For example, does taxon A have a significantly different morphology when spatially associated with taxon B?

Within a region of a given size, count the number of unique morphologies and the range of object sizes for each of the unique morphologies.

For metrics, Renyi dimension/moments can be used. This is a measure of self-similarity and scaling over different moment numbers.

### Example 11: Modeling

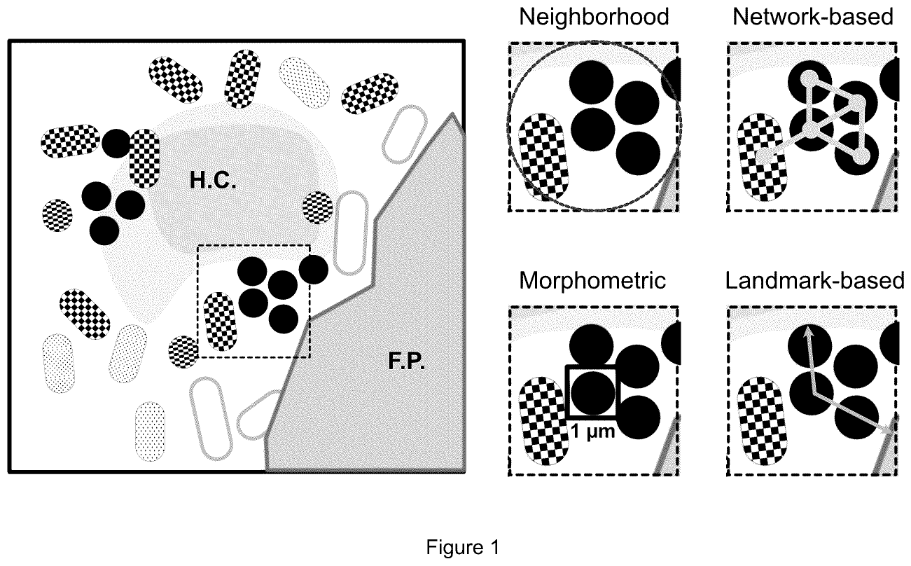

Measurements can be made using imaging-based platforms applied to microbiome samples to inform biomarker measurements. FIG. 1 shows a diagram of such measurements as imaged by microscope. An image of stool can show microbiota (colored by taxonomic identity) as well as non-microbial artifacts like host cells and food particles. An array of analyses can be done on a cell-by-cell, pixel-by-pixel, or region-by-region basis to determine neighborhood-based metrics, network-based metrics, properties of individual cells (e.g., morphological, signal based), and their distance from landmarks like host cells and food particles.

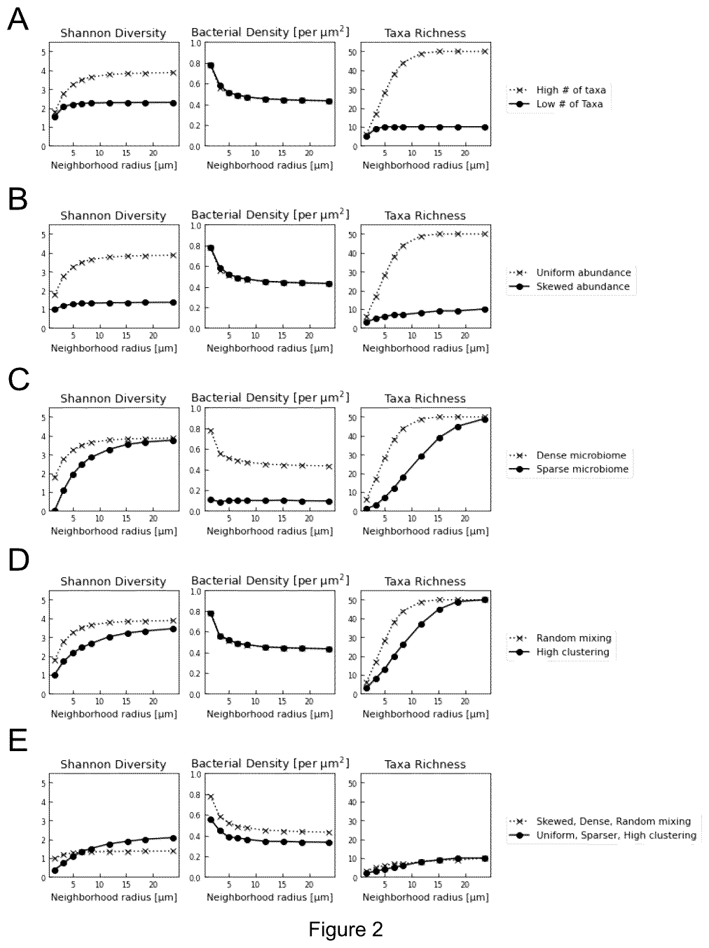

Neighborhood-based measurements for different neighborhood sizes (radius in microns) can also be modeled. FIG. 2 (panels A-E) illustrates such modeling for synthetic communities with different properties. The Shannon diversity, bacterial density (# of bacterial per square micron), and taxa richness (number of taxa) for 1000 randomly selected neighborhoods ranging in size from 1 to 30 micron radius in a 135×135 micron image of a simulated community were measured. For each metric, the median value for each neighborhood was calculated. Comparisons were made as follows: (A) microbiomes with high (n=50) and low (n=10) number of taxa, (B) uniform and power-law distributed relative abundance, (C) dense and sparse (25% of full density) microbial communities, (D) randomly mixed and clustered (using k-means clustering to create like-taxa to be nearer each other), and (E) a mixture of multiple conditions.

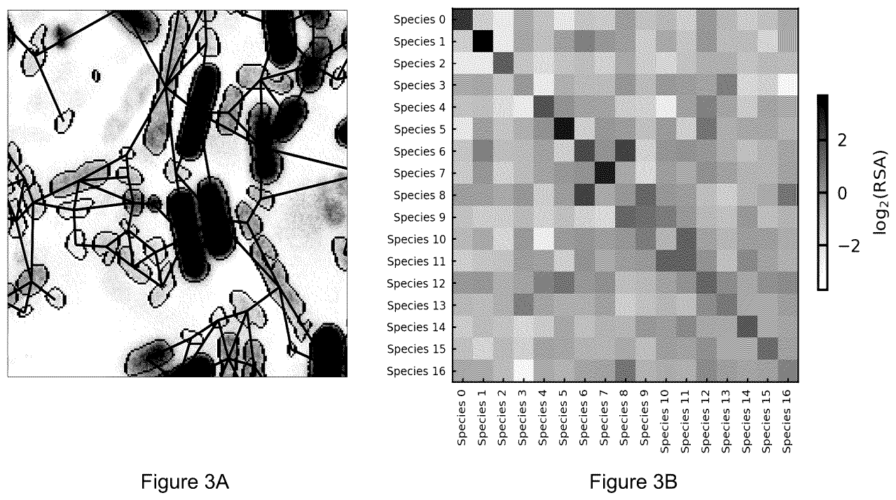

FIGS. 3A-3B demonstrate a proximity network constructed from real microbiome imaging data (zoom in is shown), a network of edges is shown superimposed over bacteria that fall within 1 micron of one another. From this proximity network and the taxonomic identification, a spatial association matrix can be constructed. By comparing the frequency of proximal associations (edges) between measured species with a large number of the frequency of proximal associations between randomly reassigned species, a spatial association matrix (right) is constructed. The relative spatial association (RSA; the fold change in measured associations over random associations) is calculated for each species. Using this approach, species interactions that are over-represented (as in Species 6 interacting with Species 8 as shown with the relatively dark gray boxes) or under-represented (as in Species 4 with Species 10 as shown with the relatively light gray boxes) are identified.

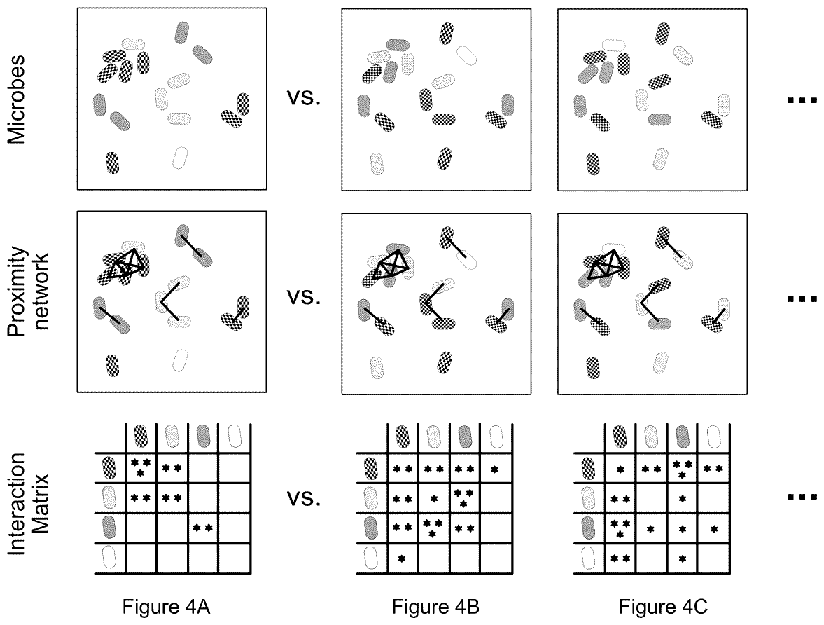

FIGS. 4A-4C diagram out a spatial association matrix and the principles associated with its construction.

### Example 12: Spatial Markers/Biomarkers

Spatial markers or biomarkers can be identified for any of the measures described above. Additionally, spatial markers or biomarkers can be identified based on average distance of specific taxa to host cells of interest (e.g., immune cells, goblet cells, enteroendocrine cells) or tissue structures of interest (e.g., Peyer's patches, lymphoid follicles, disruption of intestinal barrier).

### Example 13: Spatial Characterization in Mice

To assemble therapeutic consortia, equal-viability RCB vials were pooled under anaerobic conditions (˜6×10⁸ cells/mL), resuspended, aliquoted for storage, and thawed immediately before oral gavage into germ-free mice. Mice received the consortium 15 and 14 days before subcutaneous MCA205 tumor inoculation, followed by anti-PD-1 antibody treatments on days 6, 9, 12, and 15; tumors and feces were monitored. Tissues were harvested on day 17.

Harvested gastrointestinal tracts (stomach to rectum) were sectioned into small intestine, cecum, and colon, rinsed in ice-cold PBS, and fixed in 4% PFA for 16-24 h at 4° C. Samples were cryo-protected in a sucrose gradient (15% then 30% in PBS), embedded in OCT, frozen on dry ice, and stored at −80° C. Host-lumen boundaries in fluorescence tile scans were segmented using a fine-tuned U-Net (Segmentation Models PyTorch) with hole-filling and stray-object removal, then manually refined in Napari. Food particles were manually annotated by autofluorescence, cleaned via binary hole-filling, and validated against cryo-sectioned wheat bran controls imaged spectrally on a Zeiss LSM 980 to ensure accurate identification.

For spatial association analysis, Region Adjacency Graphs (RAGs) were built of classified cells and retained edges ≤35 px (2.45 μm) to form spatial networks. Taxon-level matrices (mSAM) were computed by tallying inter-taxon contacts and generated randomized controls (rSAM) via 1,000 label-shuffling iterations, preserving abundance and spatial constraints. Fold-change matrices (log[(mSAM+1)/(rSAM+1)]) highlighted enriched associations, with significance assessed by Bonferroni-corrected t-tests across fields. Neighborhoods were defined by packing circles (radii 25-250 μm) over tissue, recording taxon counts normalized by area, and clustering these profiles via UMAP (n_neighbors=100, min_dist=0.05). This was followed by Leiden on a k-nearest-neighbors graph (k=1500), yielding 17 distinct microbial niches; randomization experiments confirmed the non-random structure of observed spatial communities.

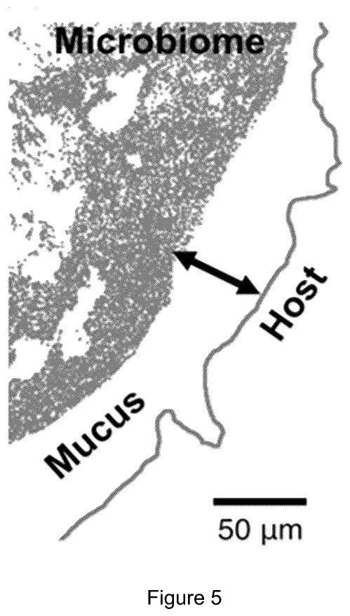

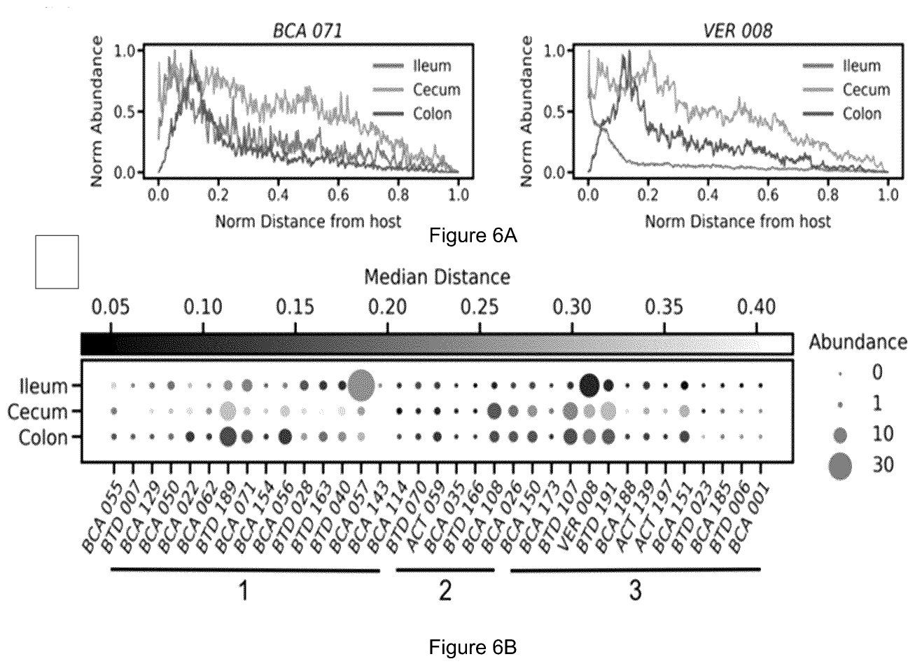

FIG. 5 shows the centroids of individual microbes near host tissue, separated by mucus layer. These centroids were used to calculate the closest distance of each microbe to the host epithelium. FIG. 6A shows the abundance distribution of two taxa relative to the host boundary in the three tissues, ileum, cecum and colon for two different strains, BCA071 and VER008. Some species show preferential enrichment near the host boundary in different regions of GI tract, while other taxa show broader distribution into the lumen. FIG. 6B depicts the median distance of each taxon from host tissue (normalized by maximum distance in that tissue) alongside their abundances in three tissue types. Bubble color represents median distance, while bubble size indicates relative abundance. The taxa on x-axis are grouped based on hierarchical clustering of median distances in all three tissue types. Three distinct groups of species (1, 2, and 3) are highlighted below the x-axis. Group 1 consisted of species that do not have preferential enrichment near the host tissue in all three regions of GI tract, Group 2 consisted of species that are enriched near the host in all three regions, and Group 3 consisted of species that are enriched near the host in ileum but not in cecum or colon.

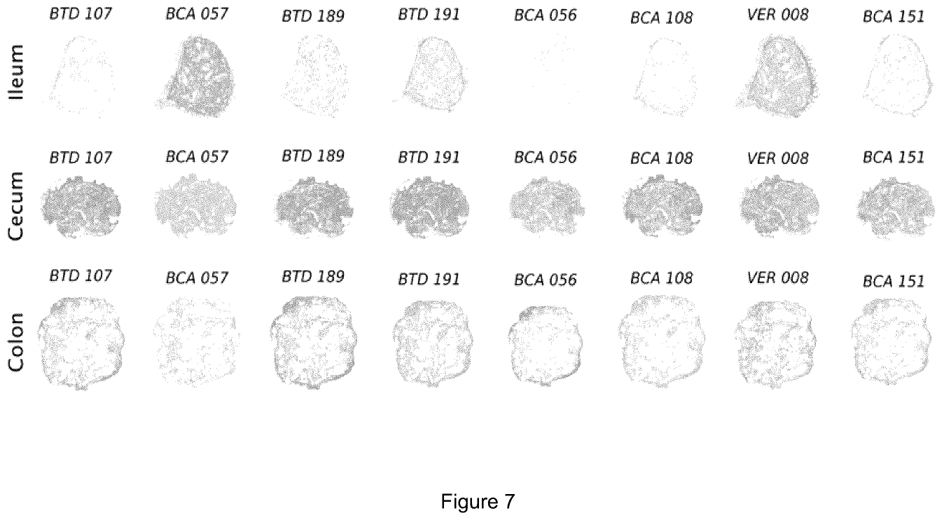

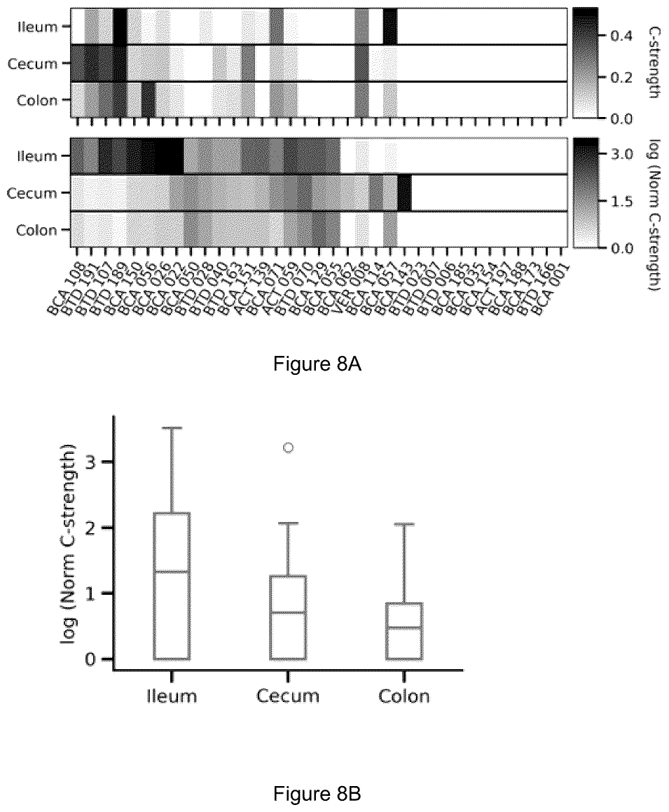

Measurement of self-interactions of different taxa were made along the GI tract. FIG. 7 details the spatial distribution of self-clusters for several individual taxa throughout the GI tract. Dark gray color indicates microbes that form clusters, while light gray represents microbes that do not. Clustering is performed for each taxon using DBSCAN, with a ˜2 μm radius defining the neighborhood around each cell and a minimum of four cells required to form a cluster. In FIG. 8A, the top heatmap image shows self-clustering strength defined by cells that form clusters normalized by absolute abundance (C-Strength) for each taxon in each tissue. This analysis provides a direct representation of species-specific self-aggregation as observed in the images. In the bottom heatmap in FIG. 8A, C-strength in the top heatmap is normalized by the C-strength measured from 5000 random simulations. Simulations are generated by randomly assigning species identities to the existing cell distributions in each tissue type while preserving their proportional abundances. Only significant values are shown (p<0.01). This analysis provides self-clustering interactions that are statistically significant. Higher C-strength indicates a higher tendency to self-aggregate. FIG. 8B shows normalized clustering strength aggregated across all strains in each region of the GI tract. The normalized clustering tendency decreased along the GI tract, which is consistent with the increasing homogenization of fecal matter as it progresses through the gut.

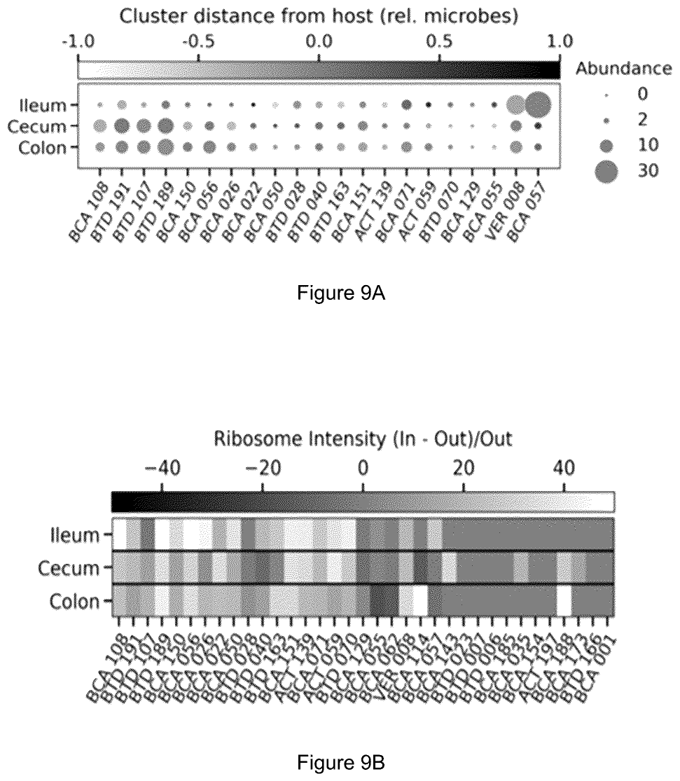

Measurement of cluster organization and ribosomal intensity along the GI tract is shown in FIGS. 9A-9B. FIG. 9A demonstrates the median distance of identified clusters of a taxon from the host tissue, normalized by the median distance of all microbes of that taxon from the host tissue. The color of the disk represents distance and the size represents abundance. Only taxa that form clusters in all three regions of GI tract are shown for comparison. A value of −1.0 indicates that the taxon forms clusters closer to the host compared to its overall distribution, while a value of +1.0 indicates that the taxon forms clusters farther from the host relative to its overall distribution. A few species, such as BCA 050, was found to preferentially form clusters closer to the host in the ileum, whereas ACT 059 tended to cluster further away in the lumen. In the cecum and colon, cluster locations were largely driven by the overall distribution of each taxon, including for BCA 050 and ACT 059. FIG. 9B shows a comparison of ribosomal intensity between cells that are within clusters and those outside of clusters, analyzed by taxon and tissue type. Lighter color indicates taxa where clustered cells have higher ribosomal intensity than non-clustered cells, while darker color indicates the opposite. The color bar extrema are capped at −50% to +50% for visualization purposes. Taxa that do not form any self-clusters are assigned a zero value. The ribosomal intensity is higher in clustered cells than in those that did not form clusters. These results suggest that cells within clusters express more ribosomes, potentially indicating higher cell activity and division within these micro-colonies.

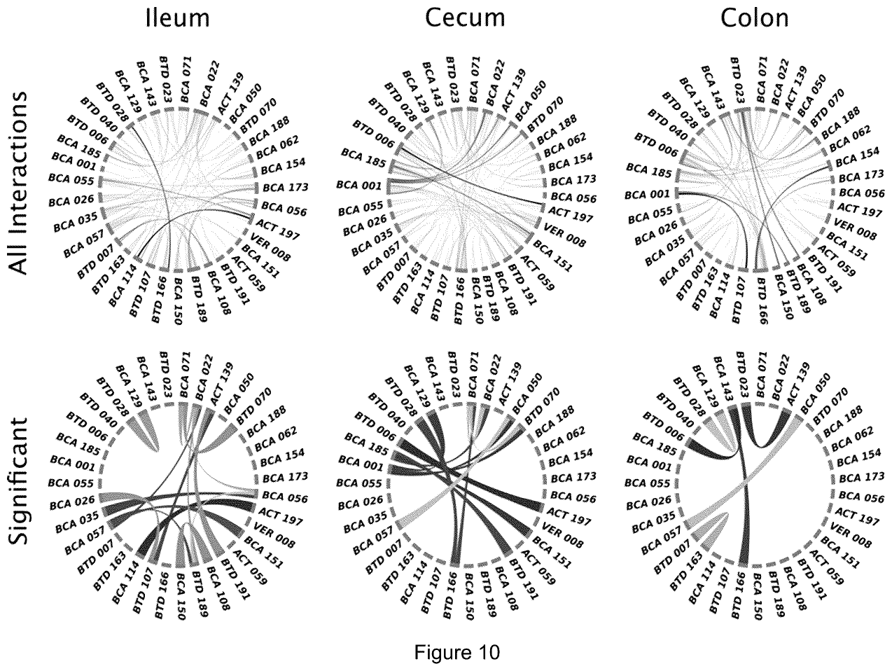

FIG. 10 details the measurement of cross-species interactions at microscale and shows non-random structures. Chord-style description shows absolute (“All Interactions” at top) and significant interactions (“Significant” at bottom) across all measured taxa in each tissue, ileum, cecum and colon. Here, self-interactions are excluded. Light gray chords show positive associations, while dark gray chords show negative associations. The significance is assessed by comparing the associations against randomized versions of the region adjacency graph. Several associations are found to be either significantly enriched or depleted. Most of the significant differential associations are tissue specific, but some associations are persistent throughout the intestine. BCA 057/BCA 050 has a significant positive spatial association across the cecum and colon; BCA 071/BCA 022 and BCA 071/BTD 070 has a significant positive association in the ileum and cecum. No persistent negative associations are observed across different tissue types, suggesting that such interactions may be primarily influenced by the local gut environment.

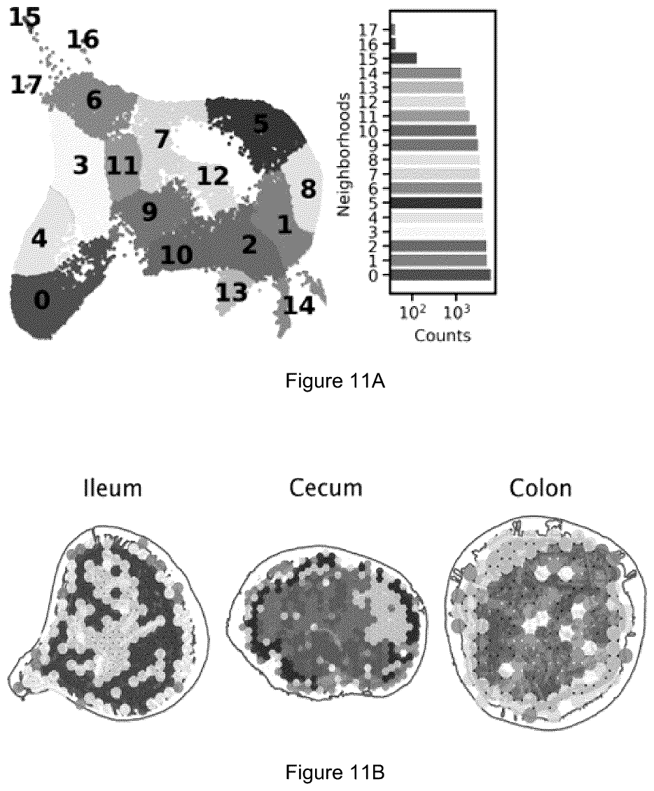

FIGS. 11A-13B relate to measurement of cross-species interactions at mesoscale and show non-random structures.

For mesoscale interactions in FIG. 11A, disks ranging from radius 25 μm to 250 μm were generated and the density of each taxon in each tissue was used to generate microbiome profiles. On the left side of FIG. 11A, the disk profiles were projected into a 2D space using UMAP and a KNN and Leiden clustering approach was used to generate distinct neighborhoods. On the right side of FIG. 11A, the frequency of each of these neighborhoods is detailed. In FIG. 111B, the distribution of neighborhoods projected onto radius-75 μm disks in each tissue is detailed. Notably, each tissue type contained a unique set of neighborhoods, highlighting the heterogeneity of spatial organization between these tissues at the mesoscale.

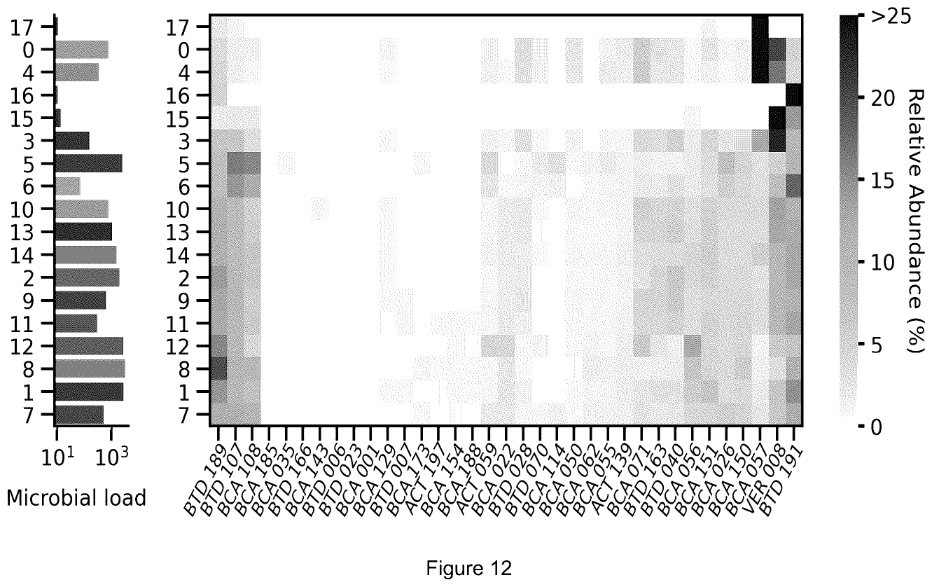

FIG. 12 shows characteristics of the neighborhoods including microbial load (taxa per 10,000 μm2) and relative abundances of different taxa.

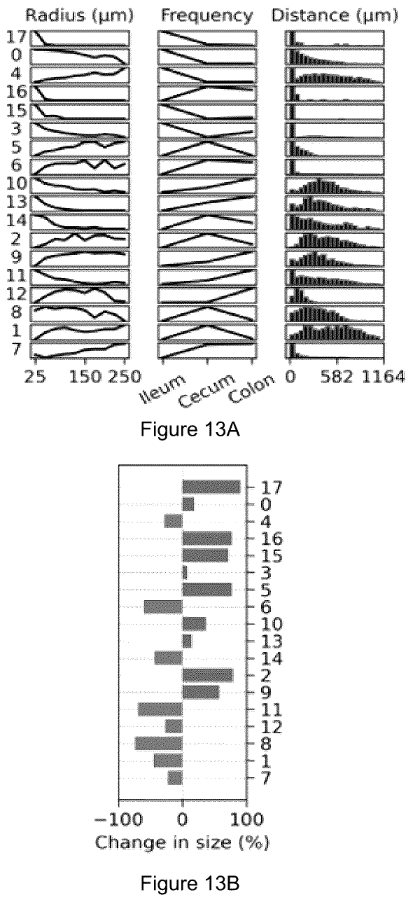

FIGS. 13A-13B detail other characteristics of the neighborhoods, including relative disk-size frequency, relative frequency per tissue, and distance from host (see FIG. 13A). As an example, Neighborhood 12 (Nbd. 12) is enriched in the colon, and consisted of many taxa, with heightened relative abundances of BTD 189 and BCA 056, and a reduced relative abundance of BTD 191 compared with several other neighborhoods. This neighborhood is distributed away from host tissue, suggesting a distinctive ecological niche. In contrast, low density neighborhoods are identified with a high relative abundance of one particular taxon with strong tissue specificity, such as BCA 057 (Nbd. 17) and VER 008 (Nbd. 15) in the ileum, and BTD 191 (Nbd. 16) in the cecum and colon. These specialized neighborhoods are exclusively observed in direct proximity to host tissue. Significance of neighborhoods over random distributions is shown in FIG. 13B. Identity randomization was used to examine neighborhood reassignment. Percentage change indicates the change in neighborhood frequency upon randomization. Strong positive values indicate neighborhoods with strong, nonrandom spatial structures.

### Example 14: Spatial Characterization in Human Fecal Samples

Samples were collected from a 30-day clinical trial in which participants received either a highly complex microbial consortium or a placebo to reduce oxalate. At multiple time points, stool specimens were harvested, immediately mixed with DNA/RNA Shield buffer, and stored at −80° C. For HiPR-Map analysis, approximately 300 μL of frozen sample was transferred under sterile conditions into a 1.5 mL tube, fixed by adding 600-900 μL of 4% paraformaldehyde, vortexing briefly, and incubating at room temperature for 15 minutes. Samples were then washed, stored in 50% ethanol, and a 0.5 μL aliquot spotted onto a microscope slide for hybridization with universal phylum-specific probes.

Mounted slides were imaged on a Zeiss i880 confocal microscope, capturing 0.07 mm² per sample. Raw images were processed by utilizing deep learning tools to automatically segment bacterial cells, classify the fluorescent dyes present in each cell, assign the corresponding phylum, and generate regional association graphs across classified cells to count taxon-taxon proximity frequency. Interaction frequencies were visualized as heatmaps, highlighting spatial relationships among the five major phyla.

In parallel, matched stool aliquots underwent standard shotgun metagenomic sequencing and analysis; wherein stool specimen microbiota were mechanically and enzymatically lysed, sequencing libraries were prepared, libraries were sequenced to generate reads, and reads were aligned against the microbiome using the popular metagenomics analysis tool, MetaPhlAn, to quantify species-level relative abundances. For direct comparison with the imaging data, those species counts were aggregated to the phylum level, enabling side-by-side evaluation of community composition by sequencing versus spatial proximity.

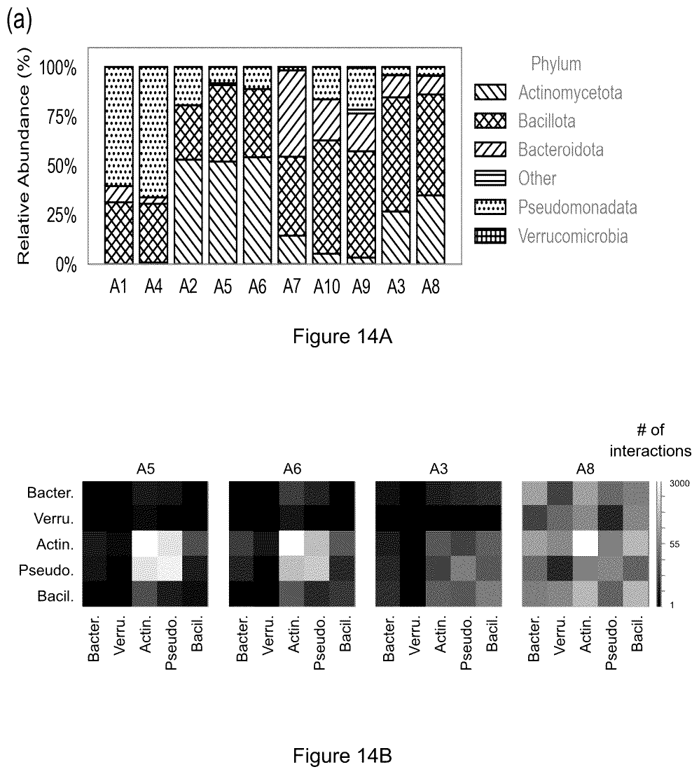

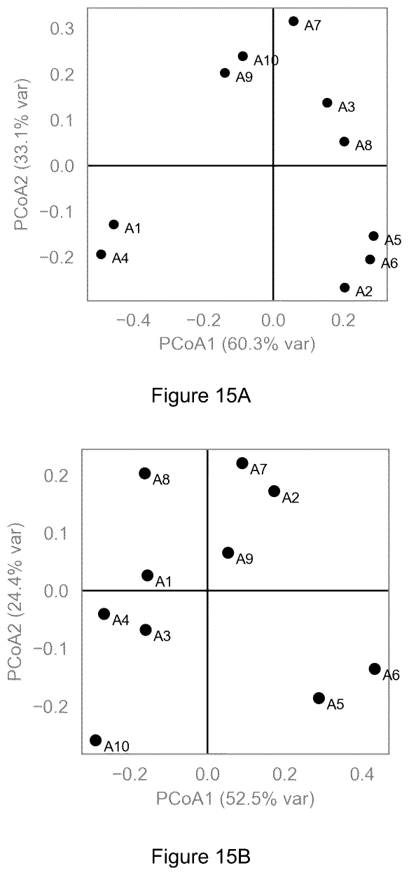

Measurements of clinical samples show differences between taxon abundance and taxon association. FIG. 14A shows metagenomic profiling of five major bacterial phyla across a clinical-trial cohort. Ten representative samples were selected for whole-metagenome sequencing and analyzed with MetaPhlAn; relative phylum abundances were then aggregated for each sample. (b) HiPR-Map fluorescence imaging targeting the same five phyla was performed on 0.07 mm² regions of each specimen as shown in FIG. 14B. Spatial adjacency graphs were constructed to count inter-phylum contacts; shading in the heatmap reflects interaction frequency for a subset of the ten samples. Principal coordinates analysis (PCoA) of the two datasets, as shown in relative-abundance profiles (FIG. 15A) and vectorized phylum-phylum interaction matrices (FIG. 15B), reveals that samples clustering by overall abundance do not always cluster similarly when spatial interaction patterns are considered.

### Example 15: Other Methods

### Whole Genome Shotgun Sequencing

Sequencing of DNA samples are carried out using the TruSeq Nano DNA Library Preparation kit (Illumina, San Diego, CA, US) and a NextSeq platform (Illumina, San Diego, CA, US). In brief, sequencing libraries are prepared from DNA extracted from each sample. DNA is mechanically fragmented using an ultrasonicator. The fragmented DNA is subjected to end repair and size selection of fragments, adenylation of 3′ ends, linked with adaptors, and DNA fragments enriched according to the TruSeq Nano DNA Library Preparation kit manual (Illumina, San Diego, CA, US). Samples are sequenced to generate more than 50 million paired-end reads of 150, 250, or 300 bp length.

16S rRNA Gene Sequencing and Species Identification

Microbial species identification is performed by full-length Sanger sequencing of the 16S rRNA gene using the 27F and 1492 primers (PMID 18296538). Species are identified by performing a bidirectional best-BLAST search against a database of curated 16S rRNA gene sequences of type species. To refine species identities, 16S rRNA gene sequences are inserted into a phylogenetic tree of curated 16S rRNA gene sequences of type species. If the sequence formed a monophyletic cluster with a known species, the strain is assigned to that species. Otherwise, the strain is assigned to a novel species. Optionally, isolates are additionally characterized by whole-genome sequencing. Genome assemblies are inserted into a phylogenetic tree of curated genomes of type species. If the sequence formed a monophyletic cluster with a known species, the strain is assigned to that species. Otherwise, the strain is assigned to a novel species.

### HiPR-FISH on Synthetic Multi-Species Microbial Communities

Cultured cells are fixed by adding an equal volume of 2% freshly made formaldehyde to the liquid culture for 1.5 hours. Fixed cells are washed 3 times in 1×PBS, permeated in absolute ethanol for 15 minutes, suspended in 50% ethanol, and stored at −20° C. until use. For each species control experiment, 1 μl of pure culture are deposited onto a UltraStick slide and air dried. To permeate cell walls, 20 μL of 10 mg/ml lysozyme suspended in 10 mM Tris-HCl is deposited onto the slide in a Frame-Seal in-situ hybridization chamber and incubated it at 370° C. for 0.5 hours. After lysozyme incubation, the slides are washed in IX PBS for 15 minutes, dipped in pure ethanol, briefly rinsed with pure ethanol to chase away any residual PBS, and air dried. The encoding hybridizations are performed in a 9×9 mm Frame-Seal hybridization chambers with 18 μl encoding hybridization buffer per slide at 460° C. for 2 hours. The slides are then washed in the Washing Buffer (215 mM NaCl, 20 mM Tris. pH 7.5, and 5 mM EDTA) at 480° C. for 15 minutes, dipped in room temperature pure ethanol, rinsed with pure ethanol, and air dried. Readout hybridizations are carried out at 460° C. for 1 hour. The slides were washed and dried as described above and embedded in 15 μl Prolong Gold Anti-fade embedding medium. For the synthetic community experiment, all bacterial suspensions are mixed together at equal volume, and 1 μL of the mixture is deposited onto an UltraStick slide and air dried. Further description can be found in US Patent Application Publication No. US2021/0047634A1 and in Shi et al., Nature 588: 676-681, 2020, each of which is incorporated by reference in its entirety.

Other embodiments are within the scope of the following claims.

What is claimed is:

1. A method of evaluating the fecal microbiome of a subject, said method comprising determining a spatial characterization of the fecal microbiome.

2. The method of claim 1, wherein determining the spatial characterization of the fecal microbiome comprises: a) imaging a fecal specimen that has been fixed, permeabilized, hybridized with encoding and readout probes, and, optionally, stained; and b) processing the imaging data to segment and characterize microbes.

3. The method of claim 1, wherein the spatial characterization of the fecal microbiome comprises measuring of one or more of microbial spatial structure, microbial spatial location, microbial intensity, microbial packing, microbial neighborhood composition, microbial interaction matrix, microbial spatial diversity, gene expression measure of microbial cell states and microbial morphology.

4. The method of claim 1, wherein the spatial characterization of the fecal microbiome is compared between a healthy subject and a subject with a disease.

5. The method of claim 1, wherein the spatial characterization of the fecal microbiome is compared between a patient who responds to treatment for a disease and a patient who does not respond to said treatment.

6. The method of claim 1, wherein the spatial characterization of the fecal microbiome is conducted in a patient to determine the appropriate treatment.

7. The method of claim 6, wherein the treatment is complementary microbiome treatment.

8. The method of claim 1, wherein the spatial characterization of the fecal microbiome is conducted to assess the efficacy of the treatment.

9. The method of claim 8, wherein the treatment is complementary microbiome treatment.

10. The method of claim 7, wherein the complementary microbiome treatment is a microbial consortium.

11. The method of claim 9, wherein the complementary microbiome treatment is a microbial consortium.

12. A method of identifying fecal microbiome biomarkers for responding to a treatment, said method comprising: a) transplanting fecal material from a treatment responder to a laboratory animal; b) allowing time for engraftment; and c) examining gastrointestinal tissue in said animal for the appearance of spatial markers as compared to control.
